(19) United States
(12) Patent Application Publication
(10) Pub. No.: US 2024/0317889 A1
(43) Pub. Date: Sep. 26, 2024

(54) ANTI-HER-2 ANTIBODY-CHEMOKINE FUSION PROTEIN, PREPARATION METHOD THEREFOR AND APPLICATION THEREOF

(71) Applicant: Shanghai Kanda Biotechnology Co., Ltd., Shanghai (CN)

(72) Inventors: Zeling Cai, Shanghai (CN); Yi Chen, Shanghai (CN)

(21) Appl. No.: 18/036,156

(22) PCT Filed: Nov. 9, 2021
(86) PCT No.: PCT/CN2021/129639

§ 371 Date: Dec. 13, 2023

(30) Foreign Application Priority Data
Nov. 10, 2020 (CN) 202011247678.1

(51) Int. Cl. C07K 16/32; A61K 35/17; A61K 38/00; A61K 47/65; A61P 35/00; C07K 14/52; C12N 5/071; C12N 15/85

(52) U.S. Cl. CPC C07K 16/32; A61K 35/17; A61K 47/65; A61P 35/00; C07K 14/523; C12N 5/0682; C12N 15/85; A61K 38/00; C07K 2319/00; C12N 2800/107

(57) ABSTRACT

Provided are an anti-Her-2 antibody-chemokine fusion protein, a preparation method therefor and an application thereof. Specifically, provided is a fusion protein, the fusion protein comprising an anti-Her-2 antibody or an active fragment thereof and a chemokine. The fusion protein of the present invention enhances the lethality of eosinophils in a tumor microenvironment by means of CCL11-mediated chemotaxis while being able to recognize tumors abnormally expressing Her-2 and inhibiting the growth thereof, further enhancing the lethality thereof toward Her-2+ tumors.

### TECHNICAL FIELD

The present invention relates to the field of biomedicine, more particularly relates to an anti-Her-2 antibody-chemokine fusion protein, preparation method therefor and application thereof.

### BACKGROUND TECHNIQUE

Her-2 is an original cancer gene, belongs to a human epidermal growth factor receptor family, inhibits cancer cells apoptosis by adjusting a downstream signal pathway, promoting proliferation and invasion of cancer cell. Her-2 amplification or overexpression accounts for about 20%-30% of breast cancer patients, and Her-2positive (Her-2+) is also normally detected in gastric cancer. The representative drug Trastuzumab of Her-2 target has good effect in breast cancer and gastric cancer of Her-2+. However, the drug resistance rate and recurrence rate of the breast cancer to the trastuzumab are increased year by year, and the final drug resistance rate is up to 65%, wherein 70% of the patients are sensitive to trastuzumab treatment at the initial stage of treatment, and finally drug resistance occurs. Therefore, there is a need for a new combination to effectively control Her-2+ tumors.

In summary, there is an urgent need in the art to develop a more safe, effective and accurate tumor targeting anti-Her-2 fusion protein.

### SUMMARY OF THE INVENTION

An object of the present invention is to provide a safe, effective and accurate tumor targeting anti-Her-2 fusion protein.

In the first aspect of the present invention, it provides a fusion protein single chain, wherein the fusion protein single chain comprises the following elements fused together:
(a) a first protein element;
(b) a second protein element; and
(c) optionally a linker element located between the first protein element and the second protein element;
wherein the first protein element is a protein element of an anti-Her-2 antibody or an active fragment thereof;
the second protein element is a protein element selected from the chemokine CC family.

In another preferred embodiment, the chemokine is selected from CCL-11 or CCL2.

In another preferred embodiment, the anti-Her-2 antibody or the active fragment thereof is an active fragment comprising F(ab), ScFv, VH, CH, VL or VHH.

In another preferred embodiment, the anti-Her-2 antibody or the active fragment thereof is selected from an active fragment of trastuzumab.

In another preferred embodiment, the linker element is a peptide bond or a peptide linker.

In the second aspect of the present invention, it provides the fusion protein composed of the fusion protein single chain of the first aspect of the present invention, and the fusion protein has a dimer structure represented by formula 1 or II:

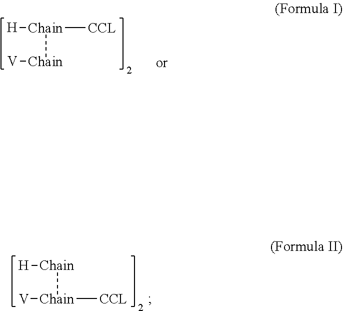

wherein,
H-Chain—V-Chain is a protein element of an anti-Her-2 antibody or an active fragment thereof, wherein,
H-Chain is none, or a heavy chain fusion protein in an anti-Her-2 antibody or an active fragment thereof;
V-Chain is none, or a light chain fusion protein in an anti-HER-2 antibody or an active fragment thereof;
CCL is a protein element selected from the chemokine CC family;
“ [symbol] ” represents a disulfide bond between a heavy chain and a light chain;
“ [symbol] ” represents a peptide bond or a peptide linker.

In another preferred embodiment, the heavy chain fusion protein comprises or contains a heavy chain, a VH, a CH, a VHH, an Fc region, or an HCDR in an anti-Her-2 antibody.

In another preferred embodiment, the light chain fusion protein comprises or contains a light chain, a VL, a CL, or an LCDR in an anti-Her-2 antibody.

In another preferred embodiment, H-Chain is a heavy chain of trastuzumab.

In another preferred embodiment, V-chain is a light chain of trastuzumab.

In another preferred embodiment, the CCL is preferably CCL-11.

In another preferred embodiment, in the fusion protein, the H-Chain or V-Chain is connected to the CCL in a header-header, head-tail, or tail-tail way.

In another preferred embodiment, the “head” refers to an N-terminal of a polypeptide or a fragment thereof, especially an N-terminal of a wild-type polypeptide or a fragment thereof.

In another preferred embodiment, the “tail” refers to a C-terminal of a polypeptide or a fragment thereof, especially a C-terminal of a wild-type polypeptide or a fragment thereof.

In another preferred embodiment, the peptide linker has a length of 0-20 amino acids, preferably 1-15 amino acids.

In another preferred embodiment, the H-chain comprises or contains positions 1-449 in SEQ ID NO: 19, the V-chain comprises or contains positions 1-214 in SEQ ID NO: 22, and CCL11 comprises or contains positions 450-523 in SEQ ID NO: 19, or positions 215-288 in SEQ ID NO: 22.

In another preferred embodiment, the sequence of the peptide linker is positions 215-221 in SEQ ID NO: 23.

In another preferred embodiment, the sequence of the fusion protein is selected from the following group:
(1) the amino acid sequence of the light chain is as shown in SEQ ID NO: 20; and the amino acid sequence of the heavy chain is as shown in SEQ ID NO.: 19.
(2) the amino acid sequence of the light chain is as shown in SEQ ID NO: 22; and the amino acid sequence of the heavy chain is as shown in SEQ ID NO.: 21; or
(3) the amino acid sequence of the light chain is as shown in SEQ ID NO: 23; and the amino acid sequence of the heavy chain is as shown in SEQ ID NO.: 21.
(4) a polypeptide derived from (1) to (3) that is formed by substitution, deletion or addition of one or more amino acid residues from (1) to (3) and has both Her-2 protein binding and CCL-11 binding activity.

In the third aspect of the invention, it provides an isolated polynucleotide, and the polynucleotide encodes the fusion protein of the second aspect of the present invention.

In another preferred embodiment, the nucleotide sequence of the fusion protein is selected from the following group:
(1) the nucleotide sequence of the light chain is as shown in SEQ ID NO: 15; and the amino acid sequence of the heavy chain is as shown in SEQ ID NO: 14.
(1) the nucleotide sequence of the light chain is as shown in SEQ ID NO: 17; and the amino acid sequence of the heavy chain is as shown in SEQ ID NO: 16; or
(1) the nucleotide sequence of the light chain is as shown in SEQ ID NO: 18; and the amino acid sequence of the heavy chain is as shown in SEQ ID NO: 16.

In the fourth aspect of the present invention, it provides a vector comprising the polynucleotide of the third aspect of the present invention.

In another preferred embodiment, the vector comprises: bacterial plasmid, phage, yeast plasmid, plant cell virus, mammalian cell virus such as adenovirus, retrovirus, or other vectors.

In the fifth aspect of the present invention, it provides a host cell, and it comprises the vector of the fourth aspect of the present invention, or having the polynucleotide of the third aspect of the present invention integrated into the genome.

In another preferred embodiment, the host cell comprises a prokaryotic cell and a eukaryotic cell.

In another preferred embodiment, the host cell comprises a mammalian cell.

In the sixth aspect of the present invention, it provides a method for generating the fusion protein of the second aspect of the present invention, and it comprises steps:
(1) culturing the host cell of the fifth aspect of the present invention under the condition suitable for expression, so as to express the fusion protein of the second aspect of the present invention; and
(2) optionally separating the fusion protein.

In the seventh aspect of the present invention, it provides a pharmaceutical composition comprising:
the fusion protein of the second aspect of the present invention, and
a pharmaceutically acceptable carrier.

In another preferred embodiment, the pharmaceutical composition further comprises: an additional active ingredient, preferably the active ingredient comprises: a small molecule compound, a cytokine, an antibody (such as an anti-PD-1 antibody, an anti-OX40 antibody, an anti-CD137 antibody, an anti-CD47 antibody, an ADC, and a CAR-immune cell.).

In another preferred embodiment, the pharmaceutical composition is in the form of injection.

In the eighth aspect of the present invention, it provides an immune cell, and the immune cell carries the fusion protein of the second aspect of the present invention.

In the ninth aspect of the present invention, it provides a pharmaceutical composition comprising:
the immune cell of the eighth aspect of the present invention, and
a pharmaceutically acceptable carrier.

In the tenth aspect of the present invention, it provides a use of the fusion protein of the second aspect of the present invention or the immune cell of the eighth aspect of the present invention, for the preparation of drugs for the treatment of tumors.

In another preferred embodiment, the tumor is Her-2 positive tumor.

In another preferred embodiment, the tumor comprises: breast cancer tumors, gastric cancer tumors, bladder cancer tumors, pancreatic cancer tumors, colorectal cancer tumors, lung cancer tumors, liver cancer tumors, melanin tumors.

In another preferred embodiment, the drug for treating tumors may be combined with another tumor immunotherapy, including but not limited to: chemotherapy, anti-CD20 mAb, anti-TIM-3 mAb, anti-LAG-3 mAb, anti-CD73 mAb, anti-CD47 mAb, anti-DLL3 mAb, anti-FRmAb mAb, anti-CTLA-4 antibody, anti-OX40 antibody, anti-CD137 antibody, anti-PD-1 antibody, PD-1/PD-L1 treatment, other immune tumor drugs, anti-angiogenic agents, radiotherapy, antibody-drug conjugates (ADC), targeted therapy, or other anti-cancer drugs.

In the eleventh aspect of the present invention, it provides an immunoconjugate comprising:
(a) the fusion protein as described above; and
(b) a coupling moiety selected from the group consisting of a detectable label, a drug, a toxin, a cytokine, a radionuclide, or an enzyme.

In another preferred embodiment, the coupling moiety is selected from the group consisting of: fluorescent or a luminescent label, a radioactive label, MRI (magnetic resonance imaging) or CT (electronic computer X-ray tomography technique) contrast agent, or an enzyme capable of producing detectable products, a radionuclide, a biotoxin, a cytokine (e.g., IL-2, etc.).

In another preferred embodiment, the immunoconjugate comprises antibody-drug conjugates (ADC).

In the twelfth aspect of the present invention, it provides a method for treating tumors, and the method comprises administering to a subject in need thereof the fusion protein, or an immunoconjugate thereof, or the pharmaceutical composition thereof, as described above.

In another preferred embodiment, the tumor comprises: breast cancer tumors, gastric cancer tumors, bladder cancer tumors, pancreatic cancer tumors, colorectal cancer tumors, lung cancer tumors, liver cancer tumors, melanin tumors.

It should be understood that within the scope of the present invention, the various technical features of the present invention above and the various technical features specifically described hereinafter (as in the examples) may be combined with each other to constitute a new or preferred technical solution. Due to space limitations, it is not repeated here.

### DESCRIPTION OF DRAWINGS

FIGS. 1A-1B show two schematic structural diagrams of an anti-Her-2 monoclonal-chemokine fusion protein embodiment of the present invention. FIG. 1A is a schematic structural diagram of trastuzumab HC-CCL11 fusion protein, and

FIG. 1B is a schematic structural diagram of trastuzumab LC-CCL11 (left) and trastuzumab LC-linker-CCL11 fusion protein (right).

FIG. 2 shows SDS-PAGE electrophoresis analysis and research of trastuzumab HC-CCL11 and trastuzumab LC-linker-CCL11 fusion protein. Wherein, lanes 1, 3 and 5 are respectively 4-12% non-reduction SDS-PAGE electrophoresis analysis of trastuzumab, trastuzumab HC-CCL11 and trastuzumab LC-linker-CCL11 fusion protein. Lanes 2, 4 and 6 are each 4-12% reduction SDS-PAGE electrophoresis analysis, respectively. Lane 4 is marked with an asterisk for single-chain HC-CCL11, and lane 6 is marked with an asterisk for single-chain LC-linker-CCL11. Lane M is a protein molecular weight standard (KDa).

FIG. 3 shows the flow cytometry analysis of trastuzumab HC-CCL11 and trastuzumab LC-linker-CCL11 fusion protein binding to the cell membranes Her-2. The human breast cancer cells BT-474 highly expressed by Her-2 are incubated with trastuzumab-CCL11 fusion protein or trastuzumab with different concentrations, and then the FITC-labeled goat anti-human IgGI Fc antibody is used to detect the antibody combined with Her-2, and the FITC fluorescence intensity is detected by using a flow cytometer.

FIG. 4 shows the flow cytometry analysis of trastuzumab HC-CCL11 and trastuzumab LC-linker-CCL11 fusion protein binding to the cell membrane receptor CCR3. The CCR3 highly expressed human embryonic lung cell MRC-5 is incubated with trastuzumab-CCL11 fusion protein or trastuzumab with different concentrations, and then the FITC-labeled goat anti-human IgG1 Fc antibody is used to detect the antibody combined with CCR3, and the FITC fluorescence intensity is detected by using a flow cytometer.

FIG. 5 shows the study of trastuzumab HC-CCL11 fusion protein to inhibit mouse melanoma cell B16 expressing human Her-2 in mouse body growth. The stable cell strain B16/Her-2 transfected with the human Her-2 gene was inoculated on the back of the mouse, after 3 days, PBS (represented by “ [symbol] ”), trastuzumab HC-CCL11 fusion protein (represented by “ [symbol] ”) and trastuzumab (represented by “ [symbol] ”) were given by tail intravenous injection, respectively, and then administration once a week for a total of 4 times. The dosage of the drug is 4 mg/kg. Tumor volume measurements were calculated as (length×width× width/2).

### DETAILED DESCRIPTION

Through extensive and intensive research, the inventor accidentally finds that (a) the anti-Her-2 antibody or the active fragment thereof and (b) the protein of the chemokine CC family are fused, and the obtained fusion protein has a synergistic effect of efficiently killing tumor cell activity and small toxic and side effects. The fusion protein of the present invention targets a tumor expressing Her-2, and is fused with a chemokine with biological activity. Specifically, the fusion protein specifically recognizes the human epidermal growth factor receptor (Her-2), and attracts and adjusts the chemokine CCL 11 of the eosinophilic granulocyte. The obtained fusion protein can specifically bind to Her-2 expressed by tumor tissue and inhibit tumor growth; and the chemokine CCL11 is transferred to a targeted tumor tissue to enhance the effect of the eosinophilic granulocyte killing tumor. Therefore, the fusion protein in the present invention can be used for the treatment of Her-2+ tumors. On this basis, the inventors have completed the present invention.

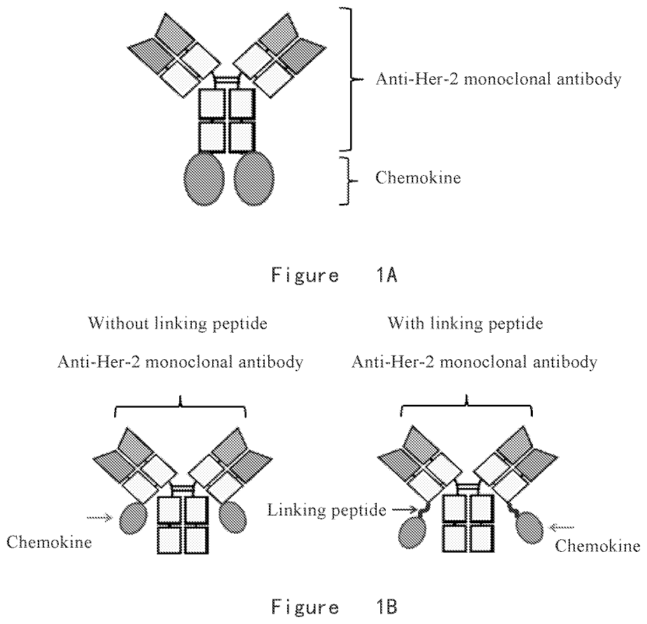

In the fusion protein designed by the present invention, as shown in FIG. 1A and FIG. 1B, chemokines may be connected to an anti-Her-2 antibody or an active fragment thereof in a head-head, head-tail, or tail-tail manner. The anti-Her-2 antibody or the active fragment thereof may be an active fragment containing F(ab), F(ab)2, scFv, or VHH, and the chemokine may be CCL 2, CCL 11. A preferred connection mode of the present invention is that the chemotactic factor CCL11 is connected to the heavy chain end of the trastuzumab. Experiments prove that the fusion protein of the present invention has a good synergistic effect, which is superior to the effect of single application of trastuzumab or chemokine CCL11, which is superior to the effect of combined use of trastuzumab and chemokine CCL11.

### The Terms

Unless otherwise defined, all technical and scientific terms used herein have the same meanings as those commonly understood by those skilled in the art to which the present invention belongs.

In the present invention, the term “fusion protein of the present invention”, “Her-2 antibody-chemokine fusion protein” and “Her-2 antibody-CCL11 fusion protein” can be used interchangeably, both referring to the fusion protein mentioned in the first aspect of the present invention.

As used herein, when used in reference to a particular recited value, the term “about” means that the value can vary by no more than 1% from the recited value. For example, as used herein, the expression “about 100” comprises all values between 99 and 101 (eg, 99.1, 99.2, 99.3, 99.4, etc.).

As used herein, unless otherwise stated, Fe refers to an Fe fragment of a human immunoglobulin. The term “immunoglobulin Fc region” refers to an immunoglobulin chain constant region, in particular a carboxyl terminal of an immunoglobulin heavy chain constant region, or a portion thereof, such as an immunoglobulin Fc region may comprise a combination of two or more domains of the heavy chain CH1, CH2, CH3 and an immunoglobulin hinge region, in preferred embodiment, the Fc region of the used immunoglobulin comprises at least one immunoglobulin stranded region, a CH2 domain and a CH3 domain, preferably lacking a CH1 domain.

Human immunoglobulins are known to have a variety of categories, such as IgA, IgD, IgE, IgM, and IgG (including IgGI, IgG2, IgG3, IgG4 subclasses), amd tje selection of a specific immunoglobulin Fc region from a specific class of immunoglobulins and subclasses is within the knowledge of those skilled in the field. In a preferred embodiment, the Fc region may be optionally contain the coding sequences of the Fc region of the IgG4 subclass of human immunoglobulin, in which an immunoglobulin heavy chain 1 domain (CH1) is missing, but the coding sequence of the hinge region as well as the CH2, CH3, and two domains are included.

As used herein, “comprising”, “having” or “including” includes “containing”, “consisting mainly of”, “consisting essentially of”, and “consisting of” “Consisting mainly of”, “consisting essentially of” and “consisting of” are subordinate concepts of “comprising”, “having” or “including”.

### Fusion Protein

As used herein, unless otherwise stated, the fusion protein is an isolated protein that is not associated with other proteins, polypeptides, or molecules, is expressed by a recombinant host cell, or a separated or purified product.

The fusion protein constructed by the present invention is composed of the following two parts:
(1) identifying a full-length monoclonal antibody or a part of a minimum recognition antigen of a tumor-specific antigen Her-2;
(2) One chemokine with biological activity in the chemotactic cytokine family such as CCL-11 or CCL2.

A fusion protein encoded ribonucleotide is constructed by using a recombinant DNA technology, and the fusion protein contains an anti-Her-2 antibody heavy chain, which contains or does not contain CH1 or CH2 or CH3 in a heavy chain constant region, and the C terminal thereof is fused with an active cell chemokine.

When the fusion protein heavy chain expression plasmid and the anti-Her-2 antibody light chain expression plasmid are co-transfected, an anti-Her-2 antibody-chemokine (such as CCL11) fusion protein can be generated, and the fusion protein can bind to tumor cells expressing Her-2 and can deliver a chemokine to a tumor site.

A chemokine with biological activity is fused with an anti-Her-2 single-chain antibody, a complete fusion protein is a polypeptide chain, each functional region is connected by a linking peptide, ensuring that the fusion protein has a correct spatial structure and maintaining its biological activity.

The fusion protein of the present invention is a brand-new molecule, and has two biological functions: first, they can target tumor tissues expressing Her-2, and second, they can specifically deliver cytokines with biological activity to tumor sites. These cytokines have the function of attracting immune cells and regulating immune cell activity, so that the tumor tissue infiltration of immune cells can be increased, the activity of immune cells can be enhanced, and tumors, such as breast cancer, gastric cancer, etc., can be inhibited. Due to the fact that chemokines are mainly limited to tumor tissue sites, the toxicity to patients is relatively small.

The antibody in the fusion protein of the present invention may be a full-length antibody, or may be a certain key segment of the antibody, for example, scFv, F(ab)2, or VHH. Theoretically, all antibodies capable of binding to the Her-2 receptor on the tumor cell membrane are suitable for constructing the antibody-chemokine fusion protein (Trastuzumab, Lapatinib monoclonal antibody, and Pertuzumab) of the present invention. In the present invention, trastuzumab is preferred.

The fusion protein chemokine moiety of the present invention is selected from chemokine of a biologically active CC family, directly or through a peptide linker to an antibody moiety.

The present invention provides a fusion protein, including the following elements:
(a) a protein element of an anti-Her-2 antibody or an active fragment thereof, (b) a protein element of a chemokine CC family (such as CCL11), and (c) a linker element. The fusion protein of the present invention may or may not contain a linker between the various elements (such as between the element A and the element B).

The fusion protein of the present invention not only has a longer in vivo half-life period, but can more effectively inhibit the concentration of antibodies (especially IgE) related to immune diseases in serum.

In the present invention, the fusion protein of the present invention further includes a conservative variant thereof, which means a polypeptide formed by replacing at most 10, preferably at most 8, more preferably at most 5, and most preferably at most 3 amino acids with amino acid of similar properties as compared with the amino acid sequence of the fusion protein of the present invention. These conservatively variant polypeptides are preferably produced by amino acid 10 substitution according to Table A.

TABLE A

| Initial residue | Representative substitution | Preferred substitution |
|---|---|---|
| Ala (A) | Val; Leu; Ile | Val |
| Arg (R) | Lys; Gln; Asn | Lys |
| Asn (N) | Gln; His; Lys; Arg | Gln |
| Asp (D) | Glu | Glu |
| Cys (C) | Ser | Ser |
| Gln (Q) | Asn | Asn |
| Glu (E) | Asp | Asp |
| Gly (G) | Pro; Ala | Ala |
| His (H) | Asn; Gln; Lys; Arg | Arg |
| Ile (I) | Leu; Val; Met; Ala; Phe | Leu |
| Leu (L) | Ile; Val; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Leu |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala | Leu |

According to the amino acid sequence provided by the present invention, those skilled in the art can conveniently prepare the fusion protein of the present invention by using various known methods. These methods are, for example, but are not limited to, recombinant DNA methods, artificial synthesis, etc. [see Murray K M, Dahl SLAnn; Pharmacother 1997 November; 31 (11): 1335-8].

After knowing the amino acid sequence of the fusion protein of the present invention, those skilled in the art can conveniently obtain the gene sequence encoding the fusion protein of the present invention according to the amino acid sequence.

A preferred fusion protein is a trastuzumab HC-CCL11 fusion protein, and the heavy chain nucleotide sequence thereof is as shown in SEQ ID NO: 14, the heavy chain amino acid sequence thereof is as shown in SEQ ID NO: 19; wherein, positions 1-449 in the heavy chain amino acid sequence (SEQ ID NO: 19) are amino acid sequence of trastuzumab; and positions 450-523 are CCL11 amino acid sequence.

A preferred fusion protein is a trastuzumab LC-CCL11 fusion protein, and the light chain nucleotide sequence thereof is as shown in SEQ ID NO: 17, the light chain amino acid sequence thereof is as shown in SEQ ID NO: 22; wherein, positions 1-214 in the light chain amino acid sequence (SEQ ID NO: 22) are light chain amino acid sequence of trastuzumab; and positions 215-288 are CCL11 amino acid sequence.

A preferred fusion protein is a trastuzumab LC-linker-CCL11 fusion protein, and the light chain nucleotide sequence thereof is as shown in SEQ ID NO: 18, the light chain amino acid sequence thereof is as shown in SEQ ID NO: 23; wherein, positions 1-214 in the light chain amino acid sequence (SEQ ID NO: 23) are light chain amino acid sequence of trastuzumab; and positions 215-221 are linker sequence; and positions 222-295 are CCL11 amino acid sequence.

In another preferred embodiment, the light chain nucleotide sequence of the trastuzumab HC-CCL11 fusion protein of the present invention is as shown in SEQ ID NO: 15, the light chain amino acid sequence thereof is as shown in SEQ ID NO: 20. In another preferred embodiment, the heavy chain nucleotide sequence of the trastuzumab LC-CCL11 or LC-linker-CCL11 fusion protein of the present invention is as shown in SEQ ID NO: 16, the heavy chain amino acid sequence thereof is as shown in SEQ ID NO: 21.

As used herein, “isolated” refers to the separation of a substance from its original environment (if it is a natural substance, the original environment is the natural environment). For example, polynucleotides and peptides in the natural state of living cells are not isolated and purified, but if the same polynucleotides or peptides are separated from other substances in the natural state, they are isolated and purified.

As used herein, “isolated recombinant fusion protein” means that the recombinant fusion protein of the present invention is substantially free of other proteins, lipids, carbohydrates or other substances naturally associated therewith. Those skilled in the art can use standard protein purification techniques to purify the recombinant fusion protein of the present invention. The substantially pure protein can produce a single main band on a non-reducing polyacrylamide gel.

The polynucleotide of the present invention can be in a form of DNA or RNA. DNA forms include cDNA, genomic DNA, or synthetic DNA. DNA can be single stranded or double stranded. DNA can be the coding strand or the non-coding strand.

The present invention also relates to variants of the above polynucleotides encoding protein fragments, analogs and derivatives of the same amino acid sequence as the present invention. The variants of this polynucleotide may be naturally occurring allelic variants or non-naturally occurring variants. These nucleotide variants include substituted variants, deletion variants, and insertion variants. As is known in the art, an allelic variant is a replacement form of a polynucleotide, which may be a substitution, deletion, or insertion of one or more nucleotides, but does not substantially change its function of encoding a polypeptide.

As used herein, the term “primer” refers to a general name for a oligonucleotide that, when paired with a template, can be used as a starting point for the synthesis of a DNA strand complementary to the template under the action of DNA polymerase. The primer may be a natural RNA, DNA, or any form of natural nucleotide. The primer may even be a non-natural nucleotide such as LNA or ZNA The primer “roughly” (or “substantially”) is complementary to a particular sequence on a chain on the template. The primer must be fully complementary to a chain on the template to begin extending, but the sequence of the primer does not have to be completely complementary to the sequence of the template. For example, at the 5′ end of the primer complementary to the template at one 3′ end plus a sequence that is not complementary to the template, such primers are still substantially complementary to the template. As long as enough long primers can be fully combined with the template, the incomplete complementary primers can also form a primer-template complex with the template, thereby performing amplification.

According to the amino acid sequence provided by the present invention, those skilled in the art can conveniently prepare the fusion protein of the present invention by using various known methods. These methods are, for example, but are not limited to, recombinant DNA methods, artificial synthesis, etc.

The nucleotide full-length sequence of the fusion protein element (such as an anti-Her-2antibody active fragment or CCL) or a fragment thereof can generally be obtained by a PCR amplification method, a recombination method or an artificial synthesis method. For PCR amplification, primers can be designed according to the disclosed related nucleotide sequences, especially open reading frame sequences, and related sequences can be obtained by using a commercially available cDNA library or a cDNA library prepared according to conventional methods known to a person skilled in the art as a template. When the sequence is long, two or more PCR amplification often needs to be performed, and then the amplified fragments are spliced together in the correct order.

Once a relevant sequence is obtained, recombination methods can be used to obtain the relevant sequence in large quantities. This is usually carried out by cloning the sequence into a vector, transforming a cell with the vector, and then separating the relevant sequence from the proliferated host cell by conventional methods.

In addition, a relevant sequence can be synthesized artificially, especially when the fragment is short in length. Usually, several small fragments are synthesized first, and then are linked together to obtain a fragment with a long sequence.

A method of amplification of DNA/RNA using PCR technology is preferred for obtaining genes of the present invention. The primers for PCR can be appropriately selected according to the sequence information of the present invention disclosed herein, and can be synthesized by a conventional method. The amplified DNA/RNA fragment can be isolated and purified by conventional methods such as by gel electrophoresis.

The present invention also includes a vector containing the polynucleotide of the present invention, and a host cell engineered by the vector or the coding sequence of the fusion protein of the present invention, and a method for producing the protein by recombination technology.

With the conventional recombinant DNA technique, the polynucleotide sequence of the present invention can be used to express or produce the recombinant protein of the present invention. Generally, the method comprises the following steps:
(1) transforming or transfecting a suitable host cell with a polynucleotide or variant thereof encoding the protein of the present invention or a recombinant expression vector containing said polynucleotide;
(2) culturing the host cell in a suitable culture medium,
(3) isolating and purifying protein from the culture medium or cell.

Method well known to the skilled in the art can be used to construct the expression vector, which contains the DNA sequence coding the protein of the invention and suitable transcription/translation control signals. These methods comprise DNA recombinant technology in vitro, DNA synthesis technology, recombinant technology in vivo, and the like. The DNA sequence can be operably linked to an appropriate promoter in an expression vector to direct mRNA synthesis. The expression vector also comprises a ribosome binding site for translation initiation and a transcription terminator.

Furthermore, the expression vector preferably comprises one or more selectable marker genes to provide phenotypic traits for selection of transformed host cells, such as dihydrofolate reductase, neomycin resistance, and green fluorescent protein (GFP) for eukaryotic cell culture, or tetracycline or ampicillin resistance for Escherichia coli.

A vector comprising the appropriate DNA sequence and the appropriate promoter or control sequence described above may be used to transform an appropriate host cell to enable it to express a protein.

The host cell can be a prokaryotic cell, such as a bacterial cell; or a lower eukaryotic cell, such as a yeast cell; or a higher eukaryotic cell, such as a mammalian cell. Representative examples are: Escherichia coli, bacterial cells of Streptomyces; fungal cells such as yeast; plant cells; insect cells of Drosophila S2 or SF9; CHO, COS, or 293 cells of animal cells, etc.

One particularly preferred cell is a cell of human and non-human mammals, particularly immune cells, including T cells, NK cells.

Transformation of host cells with recombinant DNA can be carried out using conventional techniques well known to the skilled in the art. When the host is a prokaryotic organism such as Escherichia coli, competent cells capable of absorbing DNA can be harvested after an exponential growth period and processed with a CaCl₂) method, and the steps used are well known in the art. Another method is to use MgCl2. If necessary, the transformation can also be carried out by electroporation. When the host is a eukaryote, the following DNA transfection methods can be used: calcium phosphate coprecipitation method, conventional mechanical method such as micro-injection, electroporation, liposome packaging, etc.

The obtained transformant can be cultured by conventional methods to express the polypeptide encoded by the gene of the present invention. Depending on the host cell used, the medium used in the culture may be selected from a variety of conventional medium. Culture is carried out under conditions suitable for host cell growth. After the host cell has grown to an appropriate cell density, the selected promoter is induced by a suitable method (such as temperature conversion or chemical induction) and the cells are cultured for a further period of time.

The protein in the above method may be expressed in the cell, or on the cell membrane, or secreted outside the cell. If desired, the physical, chemical and other properties can be utilized in various isolation methods to isolate and purify protein. These methods are well-known to those skilled in the art Examples of these methods include, but are not limited to, conventional renaturation treatment, treatment by protein precipitant (salt precipitation), centrifugation, cell lysis by osmosis, sonication, supercentrifugation, molecular sieve chromatography (gel chromatography), adsorption chromatography, ion exchange chromatography, high performance liquid chromatography (HPLC), and any other liquid chromatography, and the combination thereof.

### Chemokine

According to the sequence feature, the chemokine is divided into several main families, such as CC, CXC and CX3C, etc. In the present invention, the chemokine is preferentially selected from the CC family. In the CC family, CCL11 and CC2 are chemokines that are preferentially selected.

In a specific embodiment, the present invention provides an antibody-chemokine fusion protein, whose chemokine is selected from CCL11 in a CC family.

### Peptide Linker

The bifunctional fusion protein of the present invention may optionally contain or contain no peptide linker. The size and complexity of the peptide linker may affect the activity of the protein. Typically, the peptide linker should have sufficient length and flexibility to ensure that the two proteins connected have sufficient degrees of freedom in space to function its function. Meanwhile, the influence on the stability of the fusion protein such as a helix or B folding formed in the peptide linker is avoided.

The length of the peptide linker is generally 0-20 amino acids, preferably 1-15 amino acids.

Examples of preferred peptide linkers include, but are not limited to, GSGGGGS (SEQ ID NO. 24), (G4S) 3.

In a specific embodiment of the present invention, the amino acid sequence of the peptide linker is positions 215-221 in the trastuzumab LC-Linker-CCL11 amino acid sequence.

### Pharmaceutical Composition and Methods of Administration

The present invention further provides a composition, comprising (a) an effective amount of the fusion protein of the present invention or an effective amount of the immune cell of the present invention, and a pharmaceutically acceptable carrier.

Typically, the fusion protein of the present invention may be formulated in a non-toxic, inert and pharmaceutically acceptable aqueous carrier medium, wherein the pH is typically about 5-8 and preferably about 6-8.

As used herein, the term “effective amount” or “effective dose” refers to an amount that can be functional or active to humans and/or animals and that can be accepted by humans and/or animals, such as 0.001-99 wt %; preferably 0.01-95 wt %; more preferably, 0.1-90 wt %.

When the pharmaceutical composition of the present invention contains immune cells, an “effective amount” or “effective dose” refers to 1×10³ to 1×10⁷ of the immune cells/ml.

As used herein, a “pharmaceutically acceptable” component is a substance suitable for humans and/or mammals without excessive adverse side reactions (such as toxicity, stimulation, and allergy), i.e. having a reasonable benefit/risk ratio. The term “pharmaceutically acceptable carrier” refers to the carrier for using in administering the therapeutic agents, including various excipients and diluents.

The pharmaceutical composition of the present invention contains a safe and effective amount of the fusion protein of the present invention and a pharmaceutically acceptable carrier. Such carriers include, but are not limited to, saline, buffer solution, glucose, water, glycerin, ethanol or the combination thereof. In general, the pharmaceutical preparation should be matched with the administration mode, and the pharmaceutical composition of the present invention can be prepared in the form of injection, for example, prepared by a conventional method using physiological saline or an aqueous solution containing glucose and other adjuvants. The pharmaceutical composition is preferably manufactured under sterile conditions. The dosage of the active ingredient is a therapeutically effective amount. The pharmaceutical formulation of the present invention may also be made into a sustained release formulation.

The effective amount of the fusion protein of the present invention may vary with the administration mode and the severity of the disease to be treated. The selection of a preferred effective amount may be determined by one of ordinary skill in the art according to various factors (eg, by clinical trials). The factors include, but are not limited to, the pharmacokinetic parameters of the fusion protein of the present invention, such as bioavailability, metabolism, half-life period, etc.; the severity of the disease to be treated by the patient, the weight of the patient, the immune condition of the patient, the route of administration, and the like. Generally, when the fusion protein of the present invention is administered daily at a dose of about 5 mg-20 mg/kg animal weight (preferably 5 mg-10 mg/kg animal weight), a satisfactory effect can be achieved. For example, a number of separate doses may be given every day, or a dose may be proportionally reduced, by an urgent need for treatment conditions.

The fusion protein of the present invention is particularly suitable for treating diseases such as tumors. Representative tumors include (but are not limited to): breast cancer tumors, gastric cancer tumors, bladder cancer tumors, pancreatic cancer tumors, colorectal cancer tumors, lung cancer tumors, liver cancer tumors, melanin tumors.

The main advantages of the present invention are:

(1) The fusion protein of Her-2 and CCL11 of the present invention has the advantages of accurate recognition, immunotherapy, and controllable toxicity.

(2) The fusion protein of Her-2 and CCL11 of the present invention enhances the lethality of eosinophils in a tumor microenvironment by means of CCL11-mediated chemotaxis while being able to recognize tumors abnormally expressing Her-2 and inhibiting the growth thereof, further enhancing the effect thereof toward Her-2+ tumors.

(3) The fusion protein of HER-2 and CCL11 of the present invention accurately carries CCL11 to a tumor microenvironment by means of the characteristics of Her-2 targeting tumor, further strengthens the killing of eosinophilic granulocyte-mediated local tumors, and at the same time reduces the incidence of adverse reactions (eosinophilic gastroenteritis, eosinophilic trachitis, etc.) related to normal tissue eosinophilic granulocytes.

The present invention will be further illustrated below with reference to the specific examples. It is to be understood that these examples are for illustrative purposes only and are not intended to limit the scope of the invention. Those skilled in the art may make appropriate modifications and variations to the present invention, which are within the scope of the present invention.

Experimental methods that do not indicate specific conditions in the following examples may employ conventional methods in the art, such as reference to “Molecular Cloning Experiment Guide” (Third Edition, New York, Cold Spring Harbor Laboratory Press, New York: Cold Spring Harbor Laboratory Press, 1989) or according to the conditions suggested by the supplier. The sequencing method of DNA is a conventional method in the art, and may also be tested by commercial companies.

### Example 1. Construction of Antibody-CCL11 Fusion Protein Expression

### Plasmid

### 1. Construction of Trastuzumab-CCL11 Fusion Protein Expression Plasmid

Trastuzumab is an example of FIG. 1A as Her-2 antibody. The complete cDNA encoding trastuzumab heavy chain and light chain was synthesized by GenScrip (USA) company and cloned on pUC57 vector, respectively. cDNA of human CCL11 was purchased from OpenBiosystems (USA).

A large number of reports show that in the expression and preparation process of the monoclonal antibody, the heavy chain C-terminal lysine of most of the antibodies is degraded, so that when the antibody-CCL11 fusion protein was constructed, the lysine was removed, so that the antibody fusion protein of the present invention can maintain integrity.

The gene encoding the trastuzumab heavy chain and the gene encoding CCL11 were connected by a two-step polymerase chain reaction technology (PCR) method. In the first step, a heavy chain gene was amplified by a PCR method (high-fidelity polymerase Pfx, Invitrogen) with artificially synthesized antibody heavy chain DNA as a substrate:
5′ end primer M13-F (SEQ ID NO: 1): 5′-TGTAAAACGACGGCCAGT-3′, located on a pUC57 carrier.
3′ end primer KDP004 (SEQ ID NO: 2):
5′-TCCTGGGGACAGTGACAGTG-3′, which is a specific primer of the heavy chain gene of the antibody.

Likewise, genes of mature CCL11 protein moieties (Gly24-Pro97) were amplified with PCR methods:
5′ end primer KDP008 (SEQ ID NO: 3):
5′-CACTGTCACTGTCCCCAGGAGGGCCAGCTTCTGTCCCAACC-3′;
3′ end primer KDP007 (SEQ ID NO: 4):
5′-TGGTGGTGTCTAGAGACTTATGGCTTTGGAGTTGG-3′, which is a specific primer of CCL11 gene.

The nucleotide sequence of the head 20 of the primer KDP008 is complementary to the nucleotide sequence of the primer KDP004, so that the two PCR fragments can be connected in the process of the overlapping extension PCR of the second step.

After the above two PCR fragments were purified by DNA glue (Tiangen Biotech (Beijing) Co., Ltd.), the second step of overlapping PCR was performed. 5′ end primer M13-F (SEQ ID NO: 1), 3′ end primer KDP007 (SEQ ID NO: 4) contain Xba I enzyme digestion sequences for cloning.

There is a Not I enzyme digestion sites existing before the transcription start site of the trastuzumab heavy chain gene, and after the fragment obtained by overlapping PCR was purified by glue, Not I Xba I double enzyme digestion (Takara) was performed. The enzyme-digested PCR fragments were then cloned onto the same enzyme-digested mammalian cell expression vector. The mammalian cell expression vector is an improved pcDNA3.1 (Invitrogen), the anti-neomycin gene in pcDNA3.1 is substituted by a DHFR (dihydrofolate reductase) gene, and the improved vector is suitable for screening mammalian cells with high expression of stable transfection protein. The recombinant plasmid was transfected into DH5a competent bacteria, a colony PCR method was used to identify positive colonies containing correct recombinant plasmids, and the recombinant plasmids were purified. Through enzyme digestion and sequencing identification, the trastuzumab heavy chain-CCL11 recombinant gene has a correct sequence.

The trastuzumab light chain cDNA was subcloned to another modified pcDNA3.1 plasmid with the clonase Not I and XbaI.

### Example 2. Construction of Trastuzumab LC-CCL11 Fusion Protein Expression Plasmid

Trastuzumab is used as an example of a Her-2 antibody. CCL11 was directly connected to the C-terminus (LC-CCL11) of the trastuzumab light chain, or was connected to the C-terminus of the trastuzumab light chain by a linking peptide (LC-linker-CCL 11). The structure is shown in FIG. 1B.

(i) The gene encoding the trastuzumab heavy chain and the gene encoding CCL11 were connected by a two-step polymerase chain reaction technology (PCR) method. In the first step, a PCR method (high-fidelity polymerase Pfx, Invitrogen) was used to amplify light chain gene with artificially synthesized light chain DNA as a substrate:
5′ end primer KDP068 (SEQ ID NO: 5): 5′-CTTTGGCAAAGAATTGGG-3′, located on a carrier.
3′ end primer KDP206 (SEQ ID NO: 6):

5′-TCCTGGGGACAGTGACAGTG-3′, which is a specific primer of the light chain gene of the antibody.

Likewise, genes of mature CCL11 protein moieties (Gly24-Pro97) were amplified with PCR methods:
5′ end primer KDP603 (SEQ ID NO: 7):
5′-CTTTAATCGTGGCGAATGTGGGCCAGCTTCTGTC-3′,
3′ end primer BGHR (SEQ ID NO: 8):
5′-AACTAGAAGGCACAGTCGAGGC-3′, located on a carrier.

Wherein the nucleotide sequence of the head 19 of the primer KDP008 is complementary to the nucleotide sequence of the primer KDP004, so that the two PCR fragments can be connected in the process of the overlapping extension PCR of the second step.

After the above two PCR fragments were purified by DNA gel (Tiangen Biotech (Beijing) Co., Ltd.), the second step of overlapping extension PCR reaction was performed.

5′ end primer KDP066 (SEQ ID NO: 9):
5′-CGAACATCGATTGAATTCC-3′;
3′ end primer KDP605 (SEQ ID NO: 10):
5′-GAATAGGGCCCTCTAGAGACTTATGGCTTTGGAGTTG-3′

The LC-CCL11 recombinant gene was cloned into a mammalian cell expression vector by using the same method as constructing the HC-CCL11 expression gene in Example 1, and then a plasmid was prepared.

(ii) The gene encoding the trastuzumab LC-linker-CCL11 was connected by a two-step polymerase chain reaction technology (PCR) method. The method is the same as that used to construct the LC-CCL11 gene, except that the 3′ end primers of the light chain PCR and the 5′ end primers of the CCL11 PCR are different.

Light chain PCR 3′ end primer KDP602 (SEQ ID NO: 11):
5′-GATCCGCCACCGCCGCTGCCACATTCGCCACGATTAAAG-3′
CCL11 PCR 5′ end primer KDP601 (SEQ ID NO: 12):
5′-GGCAGCGGCGGTGGCGGATCCGGGCCAGCTTCTGTC-3′

The nucleotide sequence of the head 20 of the primer KDP601 is complementary to the nucleotide sequence of the head 20 of the primer KDP602, so that the two PCR fragments can be connected in the process of the overlapping extension PCR of the second step.

After the above two PCR fragments were purified by DNA glue (Tiangen Biotech (Beijing) Co., Ltd.), the second step of overlapping extension PCR reaction was performed.

The LC-linker-CCL11 recombinant gene was cloned into a mammalian cell expression vector by using the same method as constructing the HC-CCL11 expression gene in Example 1, and then plasmid was prepared.

Peptide linker nucleotide sequence (SEQ ID NO: 13):

GGCAGCGGCGGTGGCGGATCC

(iii) Trastuzumab heavy chain cDNA was cloned into mammalian cell expression vectors using a subcloning method, such as pcDNA3.1 plasmid, and the clonases were Not I and XbaI.

### Example 3. The Stable Expression Cell Strain of Trastuzumab-CCL11 Fusion Protein was Established

The host cells used to stably express trastuzumab-CCL11 fusion protein were Chinese hamster ovary cells CHO-KS. CHO-KS were CHO-K1 cells grown in fetal bovine serum (FBS) medium that were cultured with progressively reduced FBS content until FBS free, and eventually acclimated into cells grown in suspension in OptiCHO culture medium (Invitrogen) without FBS. The anti-neomycin gene in the pcDNA3.1 vector containing the fusion protein gene was substituted with rat glutamine synthetase gene, the heavy chain expression plasmid and the light chain expression plasmid were co-transfected into CHO-KS cells by adopting a method of electrotransfection (Bio-Rad, Gene Pulser Xcell), and the transfected cells were screened and cultured on the 96-well culture plate by using a limited dilution method after culturing for 24-48 hours. The screening medium was OptiCHO, 5 μg/ml recombinant human insulin and 10 μM aminosulfoxide methionine (MSX). Cells were cultured in incubator at 37° C., 8% CO₂. After three weeks, an ELISA method (a goat anti-human IgG Fc antibody coupled with an alkaline phosphatase, Jackson ImmunoResearch Lab) was used to analyze the cell culture medium of each pore with cell population, and the cell population with positive fusion protein expression was further amplified, and then, ELISA detection was performed, and further amplification was performed, and finally a fusion protein expression stable cell population was obtained.

The plasmid for expressing the trastuzumab HC-CCL fusion protein is an HC-CCL expression plasmid and an LC expression plasmid, and the plasmid for expressing the trastuzumab LC-linker-CCL11 fusion protein is an HC expression plasmid and an LC-linker-CCL11 expression plasmid.

### Example 4. Preparation, Purification and Identification of Trastuzumab-CCL11 Fusion Protein

The trastuzumab HC-CCL11 fusion protein and trastuzumab HC-linker-CCL11 fusion protein obtained in Example 3 were respectively cultured and expanded to 2 liters. The culture solution supernatant was used to purify and prepare the antibody Protein-A affinity chromatography (POROS MabCapture A, Life Tech), followed by purification with an anion column (flow through).

### Results and Analysis

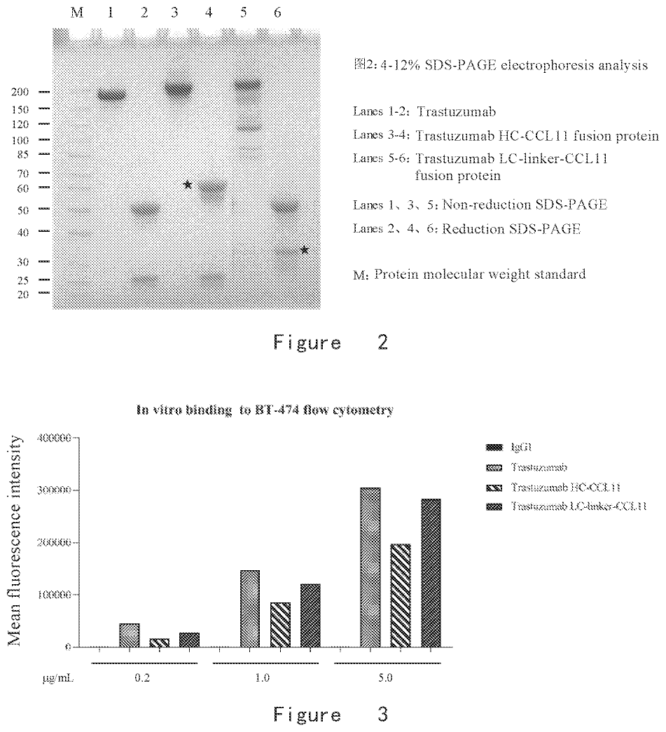

The 4-12% non-reducing SDS-PAGE electrophoretic gel of FIG. 2 (lanes 1, 3, and 5) shows that the molecular weight of the complete trastuzumab HC-CCL11 fusion protein and trastuzumab LC-linker-CCL11 fusion protein is slightly greater than that of trastuzumab, close to its theoretical value 162 kDa. 4-12% of reduced SDS-PAGE electrophoretic gel (lanes 2, 4 and 6) show that (i) the heavy chain of trastuzumab HC-CCL11 fusion protein (lane 4, asterisk) is slightly greater than trastuzumab heavy chain (lane 2), consistent with the theoretical molecular weight thereof (58 kDa), and the light chain of trastuzumab HC-CCL11 fusion protein is the same as that of trastuzumab; (ii) the light chain of the trastuzumab LC-linker-CCL11 fusion protein (lane 6, asterisk) is slightly greater than the trastuzumab light chain (lane 2), and is consistent with the theoretical molecular weight thereof (32 kDa), and the heavy chain of the trastuzumab LC-CCL11 fusion protein is the same as the heavy chain of the trastuzumab.

### Example 5. Combined Study of Trastuzumab-CCL11 Fusion Protein and Her-2

The binding of the trastuzumab-CCL11 fusion protein prepared above and the Her-2 receptor on the cell membrane was detected by flow cytometry. Human breast cancer cells BT-474 (purchased from cell libraries of Chinese Academy of Sciences) were tumor cells with high expression of Her-2. A proper amount of BT-474 cells were taken, the cell density of the cells was adjusted to 2×10⁶ cells/mL with a pre-cooled FACS working solution (PBS containing 0.1% FBS), which was divided into 100 μL/tube and closed on ice for 1 hour. The trastuzumab, trastuzumab HC-CCL11 fusion protein and trastuzumab LC-linker-CCL11 fusion protein was then diluted with FACS working solution series to 50 μg/mL, 10 μg/mL and 2 μg/mL, and a series of diluted 10 μL was added to 100 μL of cell suspension, so that the final concentrations of the antibody were 5, 1, and 0.2 μg/mL, respectively. The isotype IgGI was used as a negative control. After incubation for 30 minutes on ice, 1 mL FACS working solution was added to each cell suspension, the cells were mixed by vortex, centrifugation was performed for 5 minutes at 1200 rpm/min, supernatant was discarded, and washing was repeated once. The FITC-labeled goat anti-human IgG Fc antibody was diluted with FACS working solution 1200 (Jackson ImmunoResearch Lab), and 10 μL of antibody was added to each cell suspension, so that the final concentration was 1 μg/mL, dark, and incubated on ice for 30 minutes. After incubation was completed, 1 mL FACS working solution was added to each tube of cell suspension, the cells were subjected to vortex mixing, centrifugation was performed for 5 minutes at 1200 rpm/min, supernatant was discarded, and repeated washing was performed once. Cells were detected with flow cytometer C6 (BD Biosciences).

### Results and Analysis

A flow cytometry test result shows that the trastuzumab, the trastuzumab HC-CCL 11 fusion protein and the trastuzumab LC-linker-CCL11 fusion protein can bind to Her-2 on a cell membrane, wherein the binding capability of trastuzumab LC-linker-CCL11 fusion protein is similar to that of trastuzumab, and the cell membrane Her-2 binding capability of the trastuzumab HC-CCL11 fusion protein is slightly weaker (see FIG. 3).

Therefore, the trastuzumab-CCL11 fusion protein of different structures of the present invention substantially perfectly retains the binding characteristics with

Her-2.

### Example 6. Trastuzumab-CCL11 Fusion Protein Binds to MRC-5 Cells Expressing CCR3 In Vitro

Human embryonic lung cell MRC-5 expresses CCL11 receptor membrane protein CCR3. The binding of trastuzumab-CCL11 fusion protein to cell MRC-5 was studied by flow cytometry (MRC-5 cells were purchased from cell libraries of Chinese Academy of Sciences). The experimental method refers to the method in Example 4. The concentrations of the trastuzumab-CCL11 fusion proteins were respectively 0.2, 1 and 5 μg/mL. Trastuzumab was used as a negative control.

### Results and Analysis

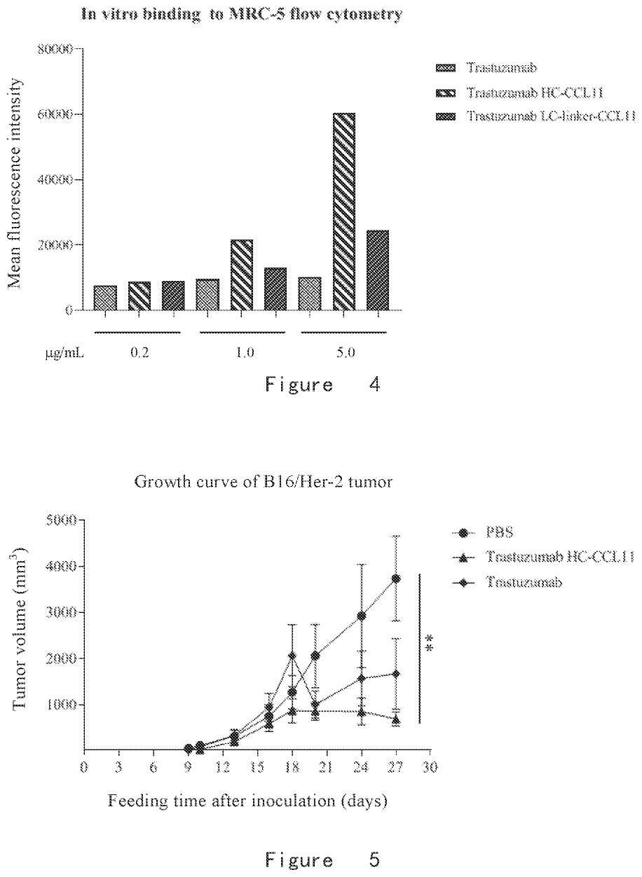

Results as shown in FIG. 4, both the trastuzumab HC-CCL11 fusion protein and the trastuzumab LC-linker-CCL11 fusion protein can bind to MRC-5, while trastuzumab does not bind to MRC-5, proving that the trastuzumab HC-CCL11 fusion protein and trastuzumab LC-linker-CCL11 fusion protein completely retain the function of specifically binding CCL11 receptor CCR3 on a cell membrane. In addition, when the trastuzumab HC-CCL11 fusion protein of the present invention is at a concentration of 5 g/mL, the ability to bind to MRC-5 cells expressing CCR3 is significantly better than that of trastuzumab LC-linker-CCL11 fusion protein and trastuzumab.

### Example 7. Construction of Mouse Tumor Cell Strain with Stable Expression of Human Her-2

Mouse melanoma cell B16 was derived from National Collection of Authenticated Cell Cultures, mouse ovarian cancer cell ID-8 was purchased from Shanghai Honsun Biological Technology Co., Ltd., and tumor cells were cultured in RPMI 1640/10% FBS (Gibco) culture medium.

Human Her-2 expression gene was cloned in expression vector pcDNA3.1 (Invitrogen), recombinant plasmid was respectively transfected into mouse melanoma cell B16 and mouse ovarian cancer cell ID-8 by Lipofectanthine 3000 (Invitrogen), and transfected cells were cultured in RPMI 10% FBS culture medium containing G418 (Sigma) to obtain the stabilized cell pool. Monoclonal stable cell strain B16/Her-2 and ID8/Her-2 with high expression of Her-2 were selected from the stable cell pool by flow cytometry (Influx, BD Biosciences).

### Example 8. The Study of Trastuzumab-CCL11 Fusion Protein Inhibits the Growth of Mouse Melanoma B16 Expressing Human her-2 in Mouse

C57BL 6 mice come from Shanghai SLAC Laboratory Animal Co., Ltd., and raised in the SPF level environment.

6-7 weeks old C57/B6 mice, groups, 8-10 mice per group, and subcutaneous inoculation were used to inject B16/Her-2 cells 3×10⁶ cells/mouse. After 2 days of cell inoculation, mice in each group were given PBS (control group), trastuzumab 4 mg/kg, CCL11 0.4 mg/kg (the same molar mass as trastuzumab), trastuzumab+CCL11 4 mg/kg (3.6 mg/kg+0.4 mg/kg, the weight percentage of CCL11 in trastuzumab-CCL11 fusion protein was 10%), trastuzumab HC-CCL11 fusion protein 4 mg/kg, trastuzumab LC-CCL11 fusion protein 4 mg/kg and trastuzumab HC-linker-CCL11 fusion protein 4 mg/kg, respectively, in the tail vein of mice in each group. The drug was then administered once a week for 4 times. The tumor volume was measured every time the drug was administered, and the weight of the mouse was weighed. Tumor volume measurements were calculated as (length×width×width/2). At the 27th day of inoculation cells, the experiment was completed, mice were euthanized by cervical dislocation, eyeballs were removed for blood collection, and mice were dissected to record tumor weight, spleen weight, and size. At the same time, tumor photos of each group were recorded. The experimental results were shown in FIG. 5 and Table 1. Table 1 shows the inhibition rate of trastuzumab HC-CCL11 fusion protein and trastuzumab on B16/Her-2tumor growth at different time points. Data indicates that the inhibition effect of trastuzumab HC-CCL11 fusion protein on tumor growth is better than that of trastuzumab.

TABLE 1
The inhibition rate of trastuzumab HC-CCL11 fusion protein and
trastuzumab on B16/Her-2 tumor growth at different time points

| | Tumor inhibitory rate | | | | |
|---|---|---|---|---|---|
| Days | D 16 | D 18 | D 20 | D 24 | D 27 |
| Trastuzumab | −25% | −62% | 51% | 46% | 55% |
| Trastuzumab | 21% | 32% | 58% | 71% | 81% |
| HC-CCL11 | | | | | |

### Results and Analysis

FIG. 5 shows that compared with a control group mouse tumor average volume, the trastuzumab HC-CCL11 fusion protein can significantly inhibit the growth of B16/Her-2 tumors in mice, and has statistical significance (P value<0.01). Trastuzumab has some impact on B16/Her-2 tumor growth, but has no statistical difference compared to the control group. In addition, each part of the trastuzumab-CCL11 fusion protein of the present invention has a synergistic effect, which is superior to that of CCL11 or trastuzumab alone, and is also superior to the combined use effect of trastuzumab and CCL11.

### DISCUSSION

Recent studies show that eosinophilic granulocytes play a significant role in inhibiting tumor growth, which can induce tumor cell death by mediating anti-tumor reaction directly and indirectly, and eosinophilic granulocytes can secrete cytotoxic protein (basic protein MBP, eosinophilic granulocyte cationic protein ECP and eosinophilic granulocyte-derived neurotoxin EDN). In addition, eosinophilic granulocytes may indirectly mediate anti-tumor effects by means of IL-12, IL-10, NK cells, IFN y, CD8+ T cells, and the like. (Sharon Grisaru-Tal, A new dawn for eosinophils in the tumour microenvironment, Nat Rev Cancer. 2020 Jul. 16) Research shows that a large amount of eosinophilic granulocyte infiltration was found in LMP pancreatic cancer tumors and BRAF melanoma models, which plays a great role in subsequent tumor immunity. (Allen B M, Systemic dysfunction and plasticity of the immune macroenvironment in cancer models. Nature Medicine, 2020: 1-10) It has been demonstrated in a large number of melanoma patients that the absolute eosinophilic granulocyte count (AEC) is positively correlated with the prognosis of melanoma patients. (Martens, A. et al. Baseline peripHeral blood biomarkers associated with clinical outcome of advanced melanoma patients treated with ipilimumab. Clin. Cancer Res. 22, 2908-2918 (2016)).

In vivo, CCL 11 plays an important role in regulating and controlling eosinophilic granulocytes. It can be mainly divided into the following two ways: firstly, inducing eosinophilic granulocytes to approach tumor microenvironment (Lorena, S Eotaxin expression in oral squamous cell carcinomas with and without tumour associated tissue eosinophilia. Oral. Dis. 9, 279-283 (2003)); secondly, enhancing the tumor killing effect mediated by eosinophilic cells. (Simson, L. Regulation of carcinogenesis by IL-5 and CCL11: a potential role for eosinophils in tumor immune surveillance. J. Immunol 178, 4222-4229 (2007)). It is observed in the colorectal cancer biopsy slice that the expression of CCL11 has a statistically positive correlation with a tumor-infiltrating eosinophilic granulocyte, and the same conclusion is also obtained in a corresponding animal model. (Eosinophils in colorectal neoplasms associated with expression of CCL11 and CCL24.J. Pathol. Transl. Med. 50, 45-51 (2016)) CCL11 in a tumor microenvironment will help the eosinophilic granulocytes to participate in immune recognition and tumor killing in a tumor microenvironment.

Related Sequence of the Present Invention

SEQ ID NO. 1
5′ end primer M13-F: TGTAAAACGACGGCCAGT
SEQ ID NO. 2
3′ end primer KDP004 TCCTGGGGACAGTGACAGTG
SEQ ID NO. 3
5′ end primer KDP008:
CACTGTCACTGTCCCCAGGAGGGCCAGCTTCTGTCCCAACC
SEQ ID NO. 4
3′ end primer KDP007:
TGGTGGTGTCTAGAGACTTATGGCTTTGGAGTTGG
SEQ ID NO. 5
5′ end primer KDP068: CTTTGGCAAAGAATTGGG
SEQ ID NO. 6
3′ end primer KDP206: ACATTCGCCACGATTAAAGGAT
SEQ ID NO. 7
5′ end primer KDP603:
CTTTAATCGTGGCGAATGTGGGCCAGCTTCTGTC
SEQ ID NO. 8
3′ end primer BGHR: AACTAGAAGGCACAGTCGAGGC
SEQ ID NO. 9
3′ end primer KDP066: CGAACATCGATTGAATTCC
SEQ ID NO. 10
3′ end primer KDP605:
GAATAGGGCCCTCTAGAGACTTATGGCTTTGGAGTTG
SEQ ID NO. 11
Light chain PCR 3′ end primer KDP602:
GATCCGCCACCGCCGCTGCCACATTCGCCACGATTAAAG
SEQ ID NO. 12 CCL11 PCR 5′ end primer KDP601:
GGCAGCGGCGGTGGCGGATCCGGGCCAGCTTCTGTC
SEQ ID NO. 13 Peptide linker nucleotide sequence:
GGCAGCGGCGGTGGCGGATCC
SEQ ID NO. 14 Heavy chain nucleotide sequence of trastuzumab HC-CCL11
fusion protein:
GAAGTGCAGCTGGTCGAATCTGGGGGAGGGCTGGTGCAGCCAGGAG
GATCACTGAGGCTGTCCTGCGCCGCTAGCGGGTTCAACATCAAGGACACC
TACATTCACTGGGTCAGACAGGCTCCTGGCAAGGGACTGGAGTGGGTGGC
ACGCATCTATCCAACTAATGGGTACACCAGATATGCCGACTCTGTGAAGG
GTCGGTTTACCATTTCTGCAGATACAAGTAAAAACACTGCCTACCTGCAG
ATGAACTCCCTGCGAGCCGAAGATACAGCCGTGTACTATTGCAGTCGTTG
GGGGGGTGACGGATTCTACGCTATGGATTATTGGGGGCAGGGCACCCTGG
TCACAGTGTCCAGCGCATCAACAAAGGGGCCTTCCGTGTTTCCACTGGCC
CCCTCTAGTAAAAGCACCTCTGGCGGAACAGCAGCCCTGGGTTGTCTGGT
GAAGGACTACTTCCCAGAGCCAGTCACCGTGTCCTGGAACAGCGGCGCCC
TGACATCCGGAGTCCATACTTTTCCTGCTGTGCTGCAGTCATCCGGGCTGT
ACAGCCTGAGCTCTGTGGTCACTGTCCCAAGTTCATCCCTGGGTACTCAG
ACCTATATCTGCAACGTGAATCACAAGCCATCCAATACCAAAGTGGACAA
GAAAGTGGAGCCCAAGAGCTGTGATAAAACACATACTTGCCCCCCTTGTC
CTGCACCAGAACTGCTGGGAGGTCCATCCGTGTTCCTGTTTCCACCCAAG
CCTAAAGACACCCTGATGATTTCTCGAACTCCAGAGGTCACCTGCGTGGT
CGTGGACGTGTCCCACGAGGACCCCGAAGTCAAGTTCAACTGGTACGTGG
ATGGCGTCGAAGTGCATAATGCTAAGACAAAACCAAGAGAGGAACAGTA
CAACAGCACTTATCGCGTCGTGTCTGTCCTGACCGTGCTGCACCAGGATT
GGCTGAACGGCAAGGAGTATAAGTGCAAAGTGAGCAATAAGGCTCTGCC
CGCACCTATCGAGAAAACAATTTCTAAGGCTAAAGGACAGCCTAGGGAA
CCACAGGTGTACACTCTGCCTCCATCTCGGGAGGAAATGACCAAGAACCA
GGTCAGTCTGACATGTCTGGTGAAAGGCTTCTATCCCTCCGACATCGCAG
TGGAGTGGGAAAGCAATGGACAGCCTGAGAACAATTACAAGACCACACC
CCCTGTGCTGGACTCTGATGGCAGTTTCTTTCTGTATAGTAAGCTGACCGT
GGATAAATCACGGTGGCAGCAGGGAAATGTCTTTAGTTGTTCAGTGATGC
ACGAAGCACTGCACAATCACTACACTCAGAAATCACTGTCACTGTCCCCA
GGAGGGCCAGCTTCTGTCCCAACCACCTGCTGCTTTAACCTGGCCAATAG
GAAGATACCCCTTCAGCGACTAGAGAGCTACAGGAGAATCACCAGTGGC
AAATGTCCCCAGAAAGCTGTGATCTTCAAGACCAAACTGGCCAAGGATAT
CTGTGCCGACCCCAAGAAGAAGTGGGTGCAGGATTCCATGAAGTATCTGG
ACCAAAAATCTCCAACTCCAAAGCCA
SEQ ID NO. 15
Light chain nucleotide sequence of trastuzumab HC-CCL11 fusion protein:
GACATTCAGATGACTCAGTCTCCTTCATCACTGTCCGCTAGCGTGGG
CGACAGAGTCACTATCACCTGCCGCGCATCCCAGGATGTGAACACCGCAG
TCGCCTGGTATCAGCAGAAGCCTGGCAAAGCTCCAAAGCTGCTGATCTAC
TCTGCAAGTTTCCTGTATAGTGGAGTGCCCTCAAGGTTTTCAGGGTCCCG
GAGCGGCACCGACTTCACACTGACTATCTCCAGCCTGCAGCCTGAGGATT
TTGCCACATACTATTGCCAGCAGCACTATACCACACCCCCTACTTTCGGCC
AGGGAACCAAAGTGGAGATCAAGCGAACTGTGGCCGCTCCATCTGTCTTC
ATTTTTCCACCCAGTGACGAACAGCTGAAGTCCGGGACAGCTAGCGTGGT
CTGTCTGCTGAACAATTTTTACCCCAGGGAAGCCAAAGTGCAGTGGAAGG
TCGATAACGCTCTGCAGTCTGGAAATAGTCAGGAGTCAGTGACAGAACA
GGACTCCAAAGATAGCACTTATTCTCTGTCTAGTACCCTGACACTGAGCA
AGGCAGACTACGAGAAGCATAAAGTGTATGCCTGTGAAGTCACTCATCA
GGGGCTGTCCAGTCCCGTCACAAAATCCTTTAATCGTGGCGAATGT
SEQ ID NO. 16
Heavy chain nucleotide sequence of trastuzumab LC-CCL11 or
LC-linker-CCL11 fusion protein
GAAGTGCAGCTGGTCGAATCTGGGGGAGGGCTGGTGCAGCCAGGAG
GATCACTGAGGCTGTCCTGCGCCGCTAGCGGGTTCAACATCAAGGACACC
TACATTCACTGGGTCAGACAGGCTCCTGGCAAGGGACTGGAGTGGGTGGC
ACGCATCTATCCAACTAATGGGTACACCAGATATGCCGACTCTGTGAAGG
GTCGGTTTACCATTTCTGCAGATACAAGTAAAAACACTGCCTACCTGCAG
ATGAACTCCCTGCGAGCCGAAGATACAGCCGTGTACTATTGCAGTCGTTG
GGGGGGTGACGGATTCTACGCTATGGATTATTGGGGGCAGGGCACCCTGG
TCACAGTGTCCAGCGCATCAACAAAGGGGCCTTCCGTGTTTCCACTGGCC
CCCTCTAGTAAAAGCACCTCTGGCGGAACAGCAGCCCTGGGTTGTCTGGT
GAAGGACTACTTCCCAGAGCCAGTCACCGTGTCCTGGAACAGCGGCGCCC
TGACATCCGGAGTCCATACTTTTCCTGCTGTGCTGCAGTCATCCGGGCTGT
ACAGCCTGAGCTCTGTGGTCACTGTCCCAAGTTCATCCCTGGGTACTCAG
ACCTATATCTGCAACGTGAATCACAAGCCATCCAATACCAAAGTGGACAA
GAAAGTGGAGCCCAAGAGCTGTGATAAAACACATACTTGCCCCCCTTGTC
CTGCACCAGAACTGCTGGGAGGTCCATCCGTGTTCCTGTTTCCACCCAAG
CCTAAAGACACCCTGATGATTTCTCGAACTCCAGAGGTCACCTGCGTGGT
CGTGGACGTGTCCCACGAGGACCCCGAAGTCAAGTTCAACTGGTACGTGG
ATGGCGTCGAAGTGCATAATGCTAAGACAAAACCAAGAGAGGAACAGTA
CAACAGCACTTATCGCGTCGTGTCTGTCCTGACCGTGCTGCACCAGGATT
GGCTGAACGGCAAGGAGTATAAGTGCAAAGTGAGCAATAAGGCTCTGCC
CGCACCTATCGAGAAAACAATTTCTAAGGCTAAAGGACAGCCTAGGGAA
CCACAGGTGTACACTCTGCCTCCATCTCGGGAGGAAATGACCAAGAACCA
GGTCAGTCTGACATGTCTGGTGAAAGGCTTCTATCCCTCCGACATCGCAG
TGGAGTGGGAAAGCAATGGACAGCCTGAGAACAATTACAAGACCACACC
CCCTGTGCTGGACTCTGATGGCAGTTTCTTTCTGTATAGTAAGCTGACCGT
GGATAAATCACGGTGGCAGCAGGGAAATGTCTTTAGTTGTTCAGTGATGC
ACGAAGCACTGCACAATCACTACACTCAGAAATCACTGTCACTGTCCCCA
GGGAAA
SEQ ID NO. 17
Light chain nucleotide sequence of trastuzumab LC-CCL11 fusion protein:
GACATTCAGATGACTCAGTCTCCTTCATCACTGTCCGCTAGCGTGGG
CGACAGAGTCACTATCACCTGCCGCGCATCCCAGGATGTGAACACCGCAG
TCGCCTGGTATCAGCAGAAGCCTGGCAAAGCTCCAAAGCTGCTGATCTAC
TCTGCAAGTTTCCTGTATAGTGGAGTGCCCTCAAGGTTTTCAGGGTCCCG
GAGCGGCACCGACTTCACACTGACTATCTCCAGCCTGCAGCCTGAGGATT
TTGCCACATACTATTGCCAGCAGCACTATACCACACCCCCTACTTTCGGCC
AGGGAACCAAAGTGGAGATCAAGCGAACTGTGGCCGCTCCATCTGTCTTC
ATTTTTCCACCCAGTGACGAACAGCTGAAGTCCGGGACAGCTAGCGTGGT
CTGTCTGCTGAACAATTTTTACCCCAGGGAAGCCAAAGTGCAGTGGAAGG
TCGATAACGCTCTGCAGTCTGGAAATAGTCAGGAGTCAGTGACAGAACA
GGACTCCAAAGATAGCACTTATTCTCTGTCTAGTACCCTGACACTGAGCA
AGGCAGACTACGAGAAGCATAAAGTGTATGCCTGTGAAGTCACTCATCA
GGGGCTGTCCAGTCCCGTCACAAAATCCTTTAATCGTGGCGAATGTGGGC
CAGCTTCTGTCCCAACCACCTGCTGCTTTAACCTGGCCAATAGGAAGATA
CCCCTTCAGCGACTAGAGAGCTACAGGAGAATCACCAGTGGCAAATGTCC
CCAGAAAGCTGTGATCTTCAAGACCAAACTGGCCAAGGATATCTGTGCCG
ACCCCAAGAAGAAGTGGGTGCAGGATTCCATGAAGTATCTGGACCAAAA
ATCTCCAACTCCAAAGCCA
SEQ ID NO. 18
Light chain nucleotide sequence of trastuzumab LC-linker-CCL11 fusion
protein:
GACATTCAGATGACTCAGTCTCCTTCATCACTGTCCGCTAGCGTGGG
CGACAGAGTCACTATCACCTGCCGCGCATCCCAGGATGTGAACACCGCAG
TCGCCTGGTATCAGCAGAAGCCTGGCAAAGCTCCAAAGCTGCTGATCTAC
TCTGCAAGTTTCCTGTATAGTGGAGTGCCCTCAAGGTTTTCAGGGTCCCG
GAGCGGCACCGACTTCACACTGACTATCTCCAGCCTGCAGCCTGAGGATT
TTGCCACATACTATTGCCAGCAGCACTATACCACACCCCCTACTTTCGGCC
AGGGAACCAAAGTGGAGATCAAGCGAACTGTGGCCGCTCCATCTGTCTTC
ATTTTTCCACCCAGTGACGAACAGCTGAAGTCCGGGACAGCTAGCGTGGT
CTGTCTGCTGAACAATTTTTACCCCAGGGAAGCCAAAGTGCAGTGGAAGG
TCGATAACGCTCTGCAGTCTGGAAATAGTCAGGAGTCAGTGACAGAACA
GGACTCCAAAGATAGCACTTATTCTCTGTCTAGTACCCTGACACTGAGCA
AGGCAGACTACGAGAAGCATAAAGTGTATGCCTGTGAAGTCACTCATCA
GGGGCTGTCCAGTCCCGTCACAAAATCCTTTAATCGTGGCGAATGTGGCA
GCGGCGGTGGCGGATCCGGGCCAGCTTCTGTCCCAACCACCTGCTGCTTT
AACCTGGCCAATAGGAAGATACCCCTTCAGCGACTAGAGAGCTACAGGA
GAATCACCAGTGGCAAATGTCCCCAGAAAGCTGTGATCTTCAAGACCAAA
CTGGCCAAGGATATCTGTGCCGACCCCAAGAAGAAGTGGGTGCAGGATT
CCATGAAGTATCTGGACCAAAAATCTCCAACTCCAAAGCCA
SEQ ID NO. 19
Heavy chain amino acid sequence of trastuzumab HC-CCL11 fusion protein:
EVQLVESGGGLVQPGGSLRLSCAASGFNIKDTYIHWVRQAPGKGLEWV
ARIYPTNGYTRYADSVKGRFTISADTSKNTAYLQMNSLRAEDTAVYYCSRW
GGDGFYAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQTYIC
NVNHKPSNTKVDKKVEPKSCDKTHTCPPCPAPELLGGPSVFLFPPKPKDTLMI
SRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSR
EEMTKNQVSLTCLVKGFYPSDIAVEWESNGQPENNYKTTPPVLDSDGSFFLY
SKLTVDKSRWQQGNVFSCSVMHEALHNHYTQKSLSLSPGGPASVPTTCCFN
LANRKIPLQRLESYRRITSGKCPQKAVIFKTKLAKDICADPKKKWVQDSMKY
LDQKSPTPKP
SEQ ID NO. 20
Light chain amino acid sequence of trastuzumab HC-CCL11 fusion protein:
DIQMTQSPSSLSASVGDRVTITCRASQDVNTAVAWYQQKPGKAPKLLIY
SASFLYSGVPSRFSGSRSGTDFTLTISSLQPEDFATYYCQQHYTTPPTFGQGTK
VEIKRTVAAPSVFIFPPSDEQLKSGTASVVCLLNNFYPREAKVQWKVDNALQ
SGNSQESVTEQDSKDSTYSLSSTLTLSKADYEKHKVYACEVTHQGLSSPVTK
SFNRGEC
SEQ ID NO. 21
Heavy chain
amino acid sequence of trastuzumab LC-CCL11 or
LC-linker-CCL11 fusion protein
EVQLVESGGGLVQPGGSLRLSCAASGFNIKDTYIHWVRQAPGKGLEWV
ARIYPTNGYTRYADSVKGRFTISADTSKNTAYLQMNSLRAEDTAVYYCSRW
GGDGFYAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQTYIC
NVNHKPSNTKVDKKVEPKSCDKTHTCPPCPAPELLGGPSVFLFPPKPKDTLMI
SRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSR
EEMTKNQVSLTCLVKGFYPSDIAVEWESNGQPENNYKTTPPVLDSDGSFFLY
SKLTVDKSRWQQGNVFSCSVMHEALHNHYTQKSLSLSPGK
SEQ ID NO. 22
Light chain amino acid sequence of trastuzumab LC-CCL11 fusion protein
DIQMTQSPSSLSASVGDRVTITCRASQDVNTAVAWYQQKPGKAPKLLIY
SASFLYSGVPSRFSGSRSGTDFTLTISSLQPEDFATYYCQQHYTTPPTFGQGTK
VEIKRTVAAPSVFIFPPSDEQLKSGTASVVCLLNNFYPREAKVQWKVDNALQ
SGNSQESVTEQDSKDSTYSLSSTLTLSKADYEKHKVYACEVTHQGLSSPVTK
SFNRGECGPASVPTTCCFNLANRKIPLQRLESYRRITSGKCPQKAVIFKTKLA
KDICADPKKKWVQDSMKYLDQKSPTPKP
SEQ ID NO. 23
Light chain amino acid sequence of trastuzumab LC-linker-CCL11 fusion
protein
DIQMTQSPSSLSASVGDRVTITCRASQDVNTAVAWYQQKPGKAPKLLIY
SASFLYSGVPSRFSGSRSGTDFTLTISSLQPEDFATYYCQQHYTTPPTFGQGTK
VEIKRTVAAPSVFIFPPSDEQLKSGTASVVCLLNNFYPREAKVQWKVDNALQ
SGNSQESVTEQDSKDSTYSLSSTLTLSKADYEKHKVYACEVTHQGLSSPVTK
SFNRGECGSGGGGSGPASVPTTCCFNLANRKIPLQRLESYRRITSGKCPQKAV
IFKTKLAKDICADPKKKWVQDSMKYLDQKSPTPKP
SEQ ID NO. 24 linker GSGGGGS

All literatures mentioned in the present application are incorporated herein by reference, as though each one is individually incorporated by reference. In addition, it should also be understood that, after reading the above teachings of the present invention, those skilled in the art can make various changes or modifications, equivalents of which falls in the scope of claims as defined in the appended claims.

1. A fusion protein single chain, wherein the fusion protein single chain comprises the following elements fused together:
(a) a first protein element;
(b) a second protein element; and
(c) optionally a linker element located between the first protein element and the second protein element;
wherein the first protein element is an anti-Her-2 antibody or a protein element of an active fragment thereof;
the second protein element is a protein element selected from the chemokine CC family.

2. The fusion protein single chain of claim 1, wherein the chemokine is selected from CCL-11 or CCL2.

3. The fusion protein single chain of claim 1, wherein the anti-Her-2 antibody or the active fragment thereof is an active fragment containing F(ab), ScFv, VH, CH, VL or VHH.

4. The fusion protein composed of the fusion protein single chain of claim 1, wherein the fusion protein has a dimer structure represented by formula I or II: 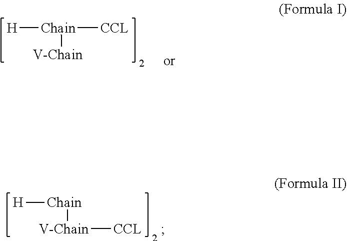
wherein,
H-Chain—V-Chain is a protein element of an anti-Her-2 antibody or an active fragment thereof, wherein,
H-chain is none, or a heavy chain fusion protein in an anti-Her-2 antibody or an active fragment thereof;
V-chain is none, or a light chain fusion protein in an anti-Her-2 antibody or an active fragment thereof;
CCL is a protein element selected from the chemokine CC family;
“|” represents a disulfide bond between a heavy chain and a light chain;
“-” represents a peptide bond or a peptide linker.

5. The fusion protein according to claim 4, wherein the sequence of the fusion protein is selected from the following group:
(1) the amino acid sequence of the light chain is as shown in SEQ ID NO: 20; and the amino acid sequence of the heavy chain is as shown in SEQ ID NO.: 19.
(2) the amino acid sequence of the light chain is as shown in SEQ ID NO: 22; and the amino acid sequence of the heavy chain is as shown in SEQ ID NO.: 21; or
(3) the amino acid sequence of the light chain is as shown in SEQ ID NO: 23; and the amino acid sequence of the heavy chain is as shown in SEQ ID NO.: 21.
(4) A polypeptide derived from (1) to (3) that is formed by substitution, deletion or addition of one or more amino acid residues from (1) to (3) and has both Her-2 protein binding and CCL-11 binding activity.

6. An isolated polynucleotide, wherein the polynucleotide encodes the fusion protein of claim 4.

7. A vector, wherein it contains the polynucleotide of claim 6.

8. A host cell, wherein it contains the vector of claim 7.

9. A method for generating the fusion protein of claim 2, wherein it comprises steps:
(1) culturing the host cell of the fusion protein under the condition suitable for expression, so as to express the fusion protein of claim 2; and
(2) optionally separating the fusion protein.

10. An immunoconjugate containing:
(a) the fusion protein of claim 4; and
(b) a coupling moiety selected from the group consisting of a detectable label, a drug, a toxin, a cytokine, a radionuclide, or an enzyme.

11. An immune cell, wherein the immune cell carries the fusion protein of claim 4.

12. A pharmaceutical composition containing:
The fusion protein of claim 4, or the immune cell expressing thereof, and
a pharmaceutically acceptable carrier.

13. The pharmaceutical composition of claim 12, wherein the pharmaceutical composition further comprises: an additional active ingredient, preferably the active ingredient comprises: a small molecule compound, a cytokine, an antibody (such as an anti-PD-1 antibody, an anti-OX40 antibody, an anti-CD137 antibody, an anti-CD47 antibody, an ADC, and a CAR-immune cell).

14.-16. (canceled)

17. A method for treating tumors, wherein the method comprises administering to a subject in need thereof the fusion protein of claim 4, or an immunoconjugate thereof, or the pharmaceutical composition thereof.

18. The method of claim 17, the tumor comprises: breast cancer tumors, gastric cancer tumors, bladder cancer tumors, pancreatic cancer tumors, colorectal cancer tumors, lung cancer tumors, liver cancer tumors, melanin tumors.


SEQUENCE LISTING (24 sequences)

SEQ ID NO: 1 - DNA, 18 bp, Artificial Sequence
  5′end primer M13-F
tgtaaaacgacggccagt

SEQ ID NO: 2 - DNA, 20 bp, Artificial Sequence
  3′end primer KDP004
tcctggggacagtgacagtg

SEQ ID NO: 3 - DNA, 41 bp, Artificial Sequence
  5′end primer KDP008
cactgtcactgtccccaggagggccagcttctgtcccaacc

SEQ ID NO: 4 - DNA, 35 bp, Artificial Sequence
  3′end primer KDP007
tggtggtgtctagagacttatggctttggagttgg

SEQ ID NO: 5 - DNA, 18 bp, Artificial Sequence
  5′end primer KDP068
ctttggcaaagaattggg

SEQ ID NO: 6 - DNA, 22 bp, Artificial Sequence
  3′end primer KDP206
acattcgccacgattaaaggat

SEQ ID NO: 7 - DNA, 34 bp, Artificial Sequence
  5′end primer KDP603
ctttaatcgtggcgaatgtgggccagcttctgtc

SEQ ID NO: 8 - DNA, 22 bp, Artificial Sequence
  3′end primer BGHR
aactagaaggcacagtcgaggc

SEQ ID NO: 9 - DNA, 19 bp, Artificial Sequence
  5′end primer KDP066
cgaacatcgattgaattcc

SEQ ID NO: 10 - DNA, 37 bp, Artificial Sequence
  3′end primer KDP605
gaatagggccctctagagacttatggctttggagttg

SEQ ID NO: 11 - DNA, 39 bp, Artificial Sequence
  3′ end primer for light chain PCR KDP602
gatccgccaccgccgctgccacattcgccacgattaaag

SEQ ID NO: 12 - DNA, 36 bp, Artificial Sequence
  5′ Primer KDP601 for CCL11 PCR
ggcagcggcggtggcggatccgggccagcttctgtc

SEQ ID NO: 13 - DNA, 21 bp, Artificial Sequence
  Peptide Linker Nucleotide Sequence
ggcagcggcggtggcggatcc

SEQ ID NO: 14 - DNA, 1569 bp, Artificial Sequence
  Nucleotide sequence of heavy chain of trastuzumab HC-CCL11 fusion protein
gaagtgcagctggtcgaatctgggggagggctggtgcagccaggaggatcactgaggctgtcctgcgccgctagcgggttcaacatcaaggacacctacattcactgggtcagacaggctcctggcaagggactggagtgggtggcacgcatctatccaactaatgggtacaccagatatgccgactctgtgaagggtcggtttaccatttctgcagatacaagtaaaaacactgcctacctgcagatgaactccctgcgagccgaagatacagccgtgtactattgcagtcgttgggggggtgacggattctacgctatggattattgggggcagggcaccctggtcacagtgtccagcgcatcaacaaaggggccttccgtgtttccactggccccctctagtaaaagcacctctggcggaacagcagccctgggttgtctggtgaaggactacttcccagagccagtcaccgtgtcctggaacagcggcgccctgacatccggagtccatacttttcctgctgtgctgcagtcatccgggctgtacagcctgagctctgtggtcactgtcccaagttcatccctgggtactcagacctatatctgcaacgtgaatcacaagccatccaataccaaagtggacaagaaagtggagcccaagagctgtgataaaacacatacttgccccccttgtcctgcaccagaactgctgggaggtccatccgtgttcctgtttccacccaagcctaaagacaccctgatgatttctcgaactccagaggtcacctgcgtggtcgtggacgtgtcccacgaggaccccgaagtcaagttcaactggtacgtggatggcgtcgaagtgcataatgctaagacaaaaccaagagaggaacagtacaacagcacttatcgcgtcgtgtctgtcctgaccgtgctgcaccaggattggctgaacggcaaggagtataagtgcaaagtgagcaataaggctctgcccgcacctatcgagaaaacaatttctaaggctaaaggacagcctagggaaccacaggtgtacactctgcctccatctcgggaggaaatgaccaagaaccaggtcagtctgacatgtctggtgaaaggcttctatccctccgacatcgcagtggagtgggaaagcaatggacagcctgagaacaattacaagaccacaccccctgtgctggactctgatggcagtttctttctgtatagtaagctgaccgtggataaatcacggtggcagcagggaaatgtctttagttgttcagtgatgcacgaagcactgcacaatcactacactcagaaatcactgtcactgtccccaggagggccagcttctgtcccaaccacctgctgctttaacctggccaataggaagataccccttcagcgactagagagctacaggagaatcaccagtggcaaatgtccccagaaagctgtgatcttcaagaccaaactggccaaggatatctgtgccgaccccaagaagaagtgggtgcaggattccatgaagtatctggaccaaaaatctccaactccaaagcca

SEQ ID NO: 15 - DNA, 642 bp, Artificial Sequence
  Nucleotide sequence of light chain of trastuzumab HC-CCL11 fusion protein
gacattcagatgactcagtctccttcatcactgtccgctagcgtgggcgacagagtcactatcacctgccgcgcatcccaggatgtgaacaccgcagtcgcctggtatcagcagaagcctggcaaagctccaaagctgctgatctactctgcaagtttcctgtatagtggagtgccctcaaggttttcagggtcccggagcggcaccgacttcacactgactatctccagcctgcagcctgaggattttgccacatactattgccagcagcactataccacaccccctactttcggccagggaaccaaagtggagatcaagcgaactgtggccgctccatctgtcttcatttttccacccagtgacgaacagctgaagtccgggacagctagcgtggtctgtctgctgaacaatttttaccccagggaagccaaagtgcagtggaaggtcgataacgctctgcagtctggaaatagtcaggagtcagtgacagaacaggactccaaagatagcacttattctctgtctagtaccctgacactgagcaaggcagactacgagaagcataaagtgtatgcctgtgaagtcactcatcaggggctgtccagtcccgtcacaaaatcctttaatcgtggcgaatgt

SEQ ID NO: 16 - DNA, 1350 bp, Artificial Sequence
  Trastuzumab LC-CCL11 or LC-linker-CCL11 fusion protein heavy chain nucleotide sequence
gaagtgcagctggtcgaatctgggggagggctggtgcagccaggaggatcactgaggctgtcctgcgccgctagcgggttcaacatcaaggacacctacattcactgggtcagacaggctcctggcaagggactggagtgggtggcacgcatctatccaactaatgggtacaccagatatgccgactctgtgaagggtcggtttaccatttctgcagatacaagtaaaaacactgcctacctgcagatgaactccctgcgagccgaagatacagccgtgtactattgcagtcgttgggggggtgacggattctacgctatggattattgggggcagggcaccctggtcacagtgtccagcgcatcaacaaaggggccttccgtgtttccactggccccctctagtaaaagcacctctggcggaacagcagccctgggttgtctggtgaaggactacttcccagagccagtcaccgtgtcctggaacagcggcgccctgacatccggagtccatacttttcctgctgtgctgcagtcatccgggctgtacagcctgagctctgtggtcactgtcccaagttcatccctgggtactcagacctatatctgcaacgtgaatcacaagccatccaataccaaagtggacaagaaagtggagcccaagagctgtgataaaacacatacttgccccccttgtcctgcaccagaactgctgggaggtccatccgtgttcctgtttccacccaagcctaaagacaccctgatgatttctcgaactccagaggtcacctgcgtggtcgtggacgtgtcccacgaggaccccgaagtcaagttcaactggtacgtggatggcgtcgaagtgcataatgctaagacaaaaccaagagaggaacagtacaacagcacttatcgcgtcgtgtctgtcctgaccgtgctgcaccaggattggctgaacggcaaggagtataagtgcaaagtgagcaataaggctctgcccgcacctatcgagaaaacaatttctaaggctaaaggacagcctagggaaccacaggtgtacactctgcctccatctcgggaggaaatgaccaagaaccaggtcagtctgacatgtctggtgaaaggcttctatccctccgacatcgcagtggagtgggaaagcaatggacagcctgagaacaattacaagaccacaccccctgtgctggactctgatggcagtttctttctgtatagtaagctgaccgtggataaatcacggtggcagcagggaaatgtctttagttgttcagtgatgcacgaagcactgcacaatcactacactcagaaatcactgtcactgtccccagggaaa

SEQ ID NO: 17 - DNA, 864 bp, Artificial Sequence
  Trastuzumab LC-CCL11 fusion protein light chain nucleotide sequence
gacattcagatgactcagtctccttcatcactgtccgctagcgtgggcgacagagtcactatcacctgccgcgcatcccaggatgtgaacaccgcagtcgcctggtatcagcagaagcctggcaaagctccaaagctgctgatctactctgcaagtttcctgtatagtggagtgccctcaaggttttcagggtcccggagcggcaccgacttcacactgactatctccagcctgcagcctgaggattttgccacatactattgccagcagcactataccacaccccctactttcggccagggaaccaaagtggagatcaagcgaactgtggccgctccatctgtcttcatttttccacccagtgacgaacagctgaagtccgggacagctagcgtggtctgtctgctgaacaatttttaccccagggaagccaaagtgcagtggaaggtcgataacgctctgcagtctggaaatagtcaggagtcagtgacagaacaggactccaaagatagcacttattctctgtctagtaccctgacactgagcaaggcagactacgagaagcataaagtgtatgcctgtgaagtcactcatcaggggctgtccagtcccgtcacaaaatcctttaatcgtggcgaatgtgggccagcttctgtcccaaccacctgctgctttaacctggccaataggaagataccccttcagcgactagagagctacaggagaatcaccagtggcaaatgtccccagaaagctgtgatcttcaagaccaaactggccaaggatatctgtgccgaccccaagaagaagtgggtgcaggattccatgaagtatctggaccaaaaatctccaactccaaagcca

SEQ ID NO: 18 - DNA, 885 bp, Artificial Sequence
  Trastuzumab LC-linker-CCL11 fusion protein light chain nucleotide sequence
gacattcagatgactcagtctccttcatcactgtccgctagcgtgggcgacagagtcactatcacctgccgcgcatcccaggatgtgaacaccgcagtcgcctggtatcagcagaagcctggcaaagctccaaagctgctgatctactctgcaagtttcctgtatagtggagtgccctcaaggttttcagggtcccggagcggcaccgacttcacactgactatctccagcctgcagcctgaggattttgccacatactattgccagcagcactataccacaccccctactttcggccagggaaccaaagtggagatcaagcgaactgtggccgctccatctgtcttcatttttccacccagtgacgaacagctgaagtccgggacagctagcgtggtctgtctgctgaacaatttttaccccagggaagccaaagtgcagtggaaggtcgataacgctctgcagtctggaaatagtcaggagtcagtgacagaacaggactccaaagatagcacttattctctgtctagtaccctgacactgagcaaggcagactacgagaagcataaagtgtatgcctgtgaagtcactcatcaggggctgtccagtcccgtcacaaaatcctttaatcgtggcgaatgtggcagcggcggtggcggatccgggccagcttctgtcccaaccacctgctgctttaacctggccaataggaagataccccttcagcgactagagagctacaggagaatcaccagtggcaaatgtccccagaaagctgtgatcttcaagaccaaactggccaaggatatctgtgccgaccccaagaagaagtgggtgcaggattccatgaagtatctggaccaaaaatctccaactccaaagcca

SEQ ID NO: 19 - protein, 523 aa, Artificial Sequence
  Trastuzumab HC-CCL11 fusion protein heavy chain amino acid sequence
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGPASVPTTCCFNLANRKIPLQRLESYRRITSGKCPQKAVIFKTKLAKDICADPKKKWVQDSMKYLDQKSPTPKP

SEQ ID NO: 20 - protein, 214 aa, Artificial Sequence
  Trastuzumab HC-CCL11 fusion protein light chain amino acid sequence
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 450 aa, Artificial Sequence
  Trastuzumab HC-CCL11 fusion protein light chain amino acid sequence
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 288 aa, Artificial Sequence
  Trastuzumab LC-CCL11 fusion protein light chain amino acid sequence
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPASVPTTCCFNLANRKIPLQRLESYRRITSGKCPQKAVIFKTKLAKDICADPKKKWVQDSMKYLDQKSPTPKP

SEQ ID NO: 23 - protein, 295 aa, Artificial Sequence
  Trastuzumab LC-linker-CCL11 fusion protein light chain amino acid sequence
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGSGGGGSGPASVPTTCCFNLANRKIPLQRLESYRRITSGKCPQKAVIFKTKLAKDICADPKKKWVQDSMKYLDQKSPTPKP

SEQ ID NO: 24 - protein, 7 aa, Artificial Sequence
  connector
GSGGGGS